(19) United States
(12) Patent Application Publication
(10) Pub. No.: US 2024/0008750 A1
(43) Pub. Date: Jan. 11, 2024

(54) Diagnosis of respiratory diseases

(71) Applicant: SANOLLA LTD., Nesher (IL)

(72) Inventors: Noa Bachner-Hinenzon, Zvia (IL); Doron Adler, Haifa (IL); David Hanuka, Haifa (IL); Eran Hadad, Hadera (IL)

(21) Appl. No.: 18/251,406

(22) PCT Filed: Oct. 21, 2021
(86) PCT No.: PCT/IB2021/059711

§ 371 Date: May 2, 2023

Related U.S. Application Data
(60) Provisional application No. 63/119,677, filed on Dec. 1, 2020.

(51) Int. Cl. A61B 5/0205; A61B 7/04; A61B 5/08; A61B 5/00; A61B 7/00; A61B 7/02

(52) U.S. Cl. CPC A61B 5/0205; A61B 7/04; A61B 5/0803; A61B 5/7257; A61B 5/7264; A61B 5/7221; A61B 5/7435; A61B 7/003; A61B 7/026; A61B 2562/0204; A61B 2562/0219; A61B 2562/0257

(57) ABSTRACT

Medical apparatus (20) includes an auscultation pickup unit (26) configured to sense acoustic waves emitted from a body of a subject (24) and to output signals in response thereto. Processing circuitry (40, 42, 44, 50) is configured to collect the signals output while the auscultation pickup unit contacts multiple locations on the body of the subject, including a respective signal acquired at each contacted location, to extract from each of the signals features of breath sounds of the subject, to compute multiple local scores including a respective local score for each contacted location based on the features extracted from the respective signal, and to classify a respiratory condition of the subject by combining the multiple local scores.

### CROSS-REFERENCE TO RELATED APPLICATION

This application claims the benefit of U.S. Provisional Patent Application 63/119,677, filed Dec. 1, 2020, which is incorporated herein by reference.

### FIELD OF THE INVENTION

The present invention relates generally to methods, apparatus, and software for medical diagnosis, and particularly to automated diagnosis of respiratory conditions.

### BACKGROUND

Auscultation has been a key technique in medical diagnosis for centuries. In auscultation, the medical practitioner listens to the internal sounds of the body, typically using a stethoscope. Auscultation is most commonly performed for the purpose of examining the circulatory and respiratory systems, and thus diagnosing conditions of the heart and lungs in particular. In more recent years, electronic stethoscopes and methods of digital processing of body sounds have become available, in order to enhance and supplement the practitioner's auditory capabilities.

PCT International Publication WO 2017/141165, whose disclosure is incorporated herein by reference, describes apparatus for detecting sound waves emanating from a body of a subject. The apparatus includes a housing and a membrane, disposed at an opening of the housing. The membrane is configured to deflect, when an outer face of the membrane contacts the body, responsively to the sound waves impinging on the membrane. The apparatus further includes a piezoelectric microphone, disposed within the housing, configured to detect vibrations of air caused by the deflection of the membrane, and to generate a microphone output in response thereto. An accelerometer, disposed on an inner face of the membrane, deflects, along with the membrane, at frequencies below a minimum frequency that is detectable by the piezoelectric microphone, and generate an accelerometer output in response thereto. A processor processes the microphone output and the accelerometer output, and generates, responsively to the processing, a sound signal that represents the impinging sound waves.

As another example, PCT International Publication WO 2019/048960, whose disclosure is incorporated herein by reference, describes a medical device, which includes a case having a front surface that is configured to be brought into contact with a body of a living subject. A microphone is contained in the case and configured to sense acoustic waves emitted from the body and to output an acoustic signal in response thereto. A proximity sensor is configured to output a proximity signal indicative of contact between the front surface and the body. At least one speaker is configured to output audible sounds. Processing circuitry is coupled to detect, in response to the proximity signal, that the front surface is in contact with the body, and in response to the detected contact, to process the acoustic signal so as to generate an audio output and to convey the audio output to the at least one speaker.

PCT International Publication WO 2019/048961, whose disclosure is incorporated herein by reference, describes diagnosis of pathologies using infrasonic signatures. In one embodiment, a medical device includes an acoustic transducer, which is configured to sense infrasonic waves emitted from a body of a living subject with a periodicity determined by a periodic physiological activity and to output an electrical signal in response to the sensed waves. At least one speaker is configured to output audible sounds in response to an electrical input. Processing circuitry is configured to process the electrical signal so as to generate a frequency-stretched signal in which infrasonic frequency components of the electrical input are shifted to audible frequencies while preserving the periodicity of the periodic physiological activity in the frequency-stretched signal, and to input the frequency-stretched signal to the at least one speaker.

### SUMMARY

Embodiments of the present invention that are described hereinbelow provide improved systems and methods for medical diagnosis.

There is therefore provided, in accordance with an embodiment of the invention, medical apparatus, including an auscultation pickup unit configured to sense acoustic waves emitted from a body of a subject and to output signals in response thereto. Processing circuitry is configured to collect the signals output while the auscultation pickup unit contacts multiple locations on the body of the subject, including a respective signal acquired at each contacted location, to extract from each of the signals features of breath sounds of the subject, to compute multiple local scores including a respective local score for each contacted location based on the features extracted from the respective signal, and to classify a respiratory condition of the subject by combining the multiple local scores.

In some embodiment, the apparatus includes a user interface, wherein the processing circuitry is configured to drive the user interface so as to guide an operator of the apparatus in placing the auscultation pickup unit in contact with each of the multiple locations. In a disclosed embodiment, the user interface includes a display screen, which is configured to display icons representing the multiple locations and indicating a status of collection of the signals from each of the multiple locations. Additionally or alternatively, the auscultation pickup unit includes a contact sensor, which is configured to output a contact signal indicative of contact between the auscultation pickup unit and the body, and the processing circuitry is configured to assess a quality of the contact responsively to the electrical signal and to prompt the operator to modify the contact between the auscultation pickup unit and the body so as to improve the quality of the contact. Further additionally or alternatively, the multiple locations include four locations on a back of the subject, including upper right, lower right, upper left, and lower left locations.

In a disclosed embodiment, the auscultation pickup unit is configured to output the signals in response to both audible and infrasonic acoustic waves emitted from the body. Additionally or alternatively, the auscultation pickup unit includes a motion sensor, which is configured to output a motion signal indicative of movement of the auscultation pickup unit, and the processing circuitry is configured to identify a respiratory cycle of the subject responsively to the motion signal and to apply the identified respiratory cycle in extracting the features. Further additionally or alternatively, the processing circuitry is configured to identify a heart rate of the subject responsively to the signals and to apply the identified heart rate in extracting the features.

In a disclosed embodiment, the auscultation pickup unit includes a first acoustic transducer, which is configured to output a first signal in response to the acoustic waves emitted from the body, and a second acoustic transducer, which is configured to output a second signal in response to ambient acoustic waves that are incident on the auscultation pickup unit, and the processing circuitry is configured to extract the features of the breath sounds responsively to a difference between the first and second signals. In one embodiment, the processing circuitry is configured to collect the first and second signals while the subject vocalizes one or more predefined sounds, and to apply the collected first and second signals in extracting the features responsively to the vocalized sounds.

In some embodiments, the extracted features include time-domain parameters and frequency-domain parameters of the digital signals. In one embodiment, the processing circuitry is configured to compute the frequency-domain parameters for each frequency among a first plurality of audible frequencies and a second plurality of infrasonic frequencies.

Additionally or alternatively, the processing circuitry is configured to classify the respiratory condition as positive, negative, or inconclusive with respect to a respiratory illness, for example with respect to COVID-19.

There is also provided, in accordance with an embodiment of the invention, a method for medical diagnosis, which includes sensing acoustic waves emitted from each of multiple locations on a body of a subject using an auscultation pickup unit, which contact each of the locations, and outputting respective signals in response thereto, including a respective signal acquired at each contacted location. Features of breath sounds of the subject are extracted from the signals. Multiple local scores are computed, including a respective local score for each contacted location based on the features extracted from the respective signal. A respiratory condition of the subject is classified by combining the multiple local scores.

The present invention will be more fully understood from the following detailed description of the embodiments thereof, taken together with the drawings in which:

### BRIEF DESCRIPTION OF THE DRAWINGS

FIG. 1 is a schematic pictorial illustration showing an electronic stethoscope in clinical use, in accordance with an embodiment of the invention;

FIG. 2 is a block diagram that schematically shows elements of an electronic stethoscope, in accordance with an embodiment of the invention;

FIG. 3 is a schematic frontal view of a display on an electronic stethoscope during a computer-guided diagnostic test, in accordance with an embodiment of the invention;

FIG. 4 is a flow chart that schematically illustrates a method for automated diagnosis of respiratory conditions, in accordance with an embodiment of the invention; and

FIGS. 5A, 5B and 5C are schematic frontal views of a display on an electronic stethoscope showing different results of diagnostic tests carried out using the electronic stethoscope, in accordance with an embodiment of the invention.

### DETAILED DESCRIPTION OF EMBODIMENTS

### Overview

Infectious respiratory diseases, such as the worldwide COVID-19 epidemic, are a matter of ongoing concern. There is a need for rapid and reliable screening and diagnosis, which cannot always be provided by chemical tests. When a patient presents with respiratory symptoms, it is necessary to determine whether the patient is in the early stages of a serious infectious illness—and if so, to identify the illness. Once a patient has been diagnosed with a potentially threatening condition, such as COVID-19 or pneumonia, it is important to be able to quickly identify and treat any deterioration in the patient's condition. Most medical personnel, however, do not have the necessary training and experience to make such a diagnosis on their own.

Embodiments of the present invention that are described herein provide apparatus and methods for automated acquisition and processing of vital signs, and particularly acoustic waves captured by auscultation, in order to classify a patient's respiratory condition. These devices and methods can be used, for example, in assessing whether the patient is suffering from a bacterial or viral respiratory infection, such as COVID-19. In a disclosed embodiment, the apparatus guides a medical caregiver in acquiring signals at a certain set of locations on the patient's back, and then processes the signals at each location and combines the results to classify the respiratory condition. Alternatively or additionally, however, measurements may be made at other locations on the patient's back or chest.

By automating the process of signal acquisition and diagnosis, the present embodiments enable rapid, reliable screening and diagnosis, even outside the clinical setting, using compact, inexpensive equipment that can be operated by personnel with only minimal medical skills. These embodiments are thus useful both in reducing contagion, by early diagnosis of infections, and in reducing unnecessary hospitalization and pressure on clinical personnel, facilities, and resources.

The embodiments that are described hereinbelow provide medical apparatus comprising an auscultation pickup unit, i.e., an electronic stethoscope head, which contacts the body of a subject and senses acoustic waves emitted from the body. The auscultation pickup unit outputs signals in response to the acoustic waves, typically including both audible and infrasonic waves. The apparatus may include a user interface, such as a display screen, which guides an operator of the apparatus in placing the auscultation pickup unit in contact with multiple locations on the subject's body.

Processing circuitry collects and digitizes the signals output by the auscultation pickup unit at each of these locations, and extracts features of the breath sounds of the subject from the signals captured at each location. On the basis of the features of the signals at each location, the processing circuitry computes a respective local score for the location. The processing circuitry then classifies the subject's respiratory condition by combining the multiple local scores. The computation of multiple local scores at multiple different locations is useful in differentiating between respiratory illnesses, as some illnesses have different effects in different areas of the lungs, as well as in improving the stability and reliability of diagnostic results. At the conclusion of the computations, the processing circuitry typically classifies the patient's respiratory condition as being positive, negative, or inconclusive with respect to one or more respiratory illnesses, for example COVID-19.

### System Description

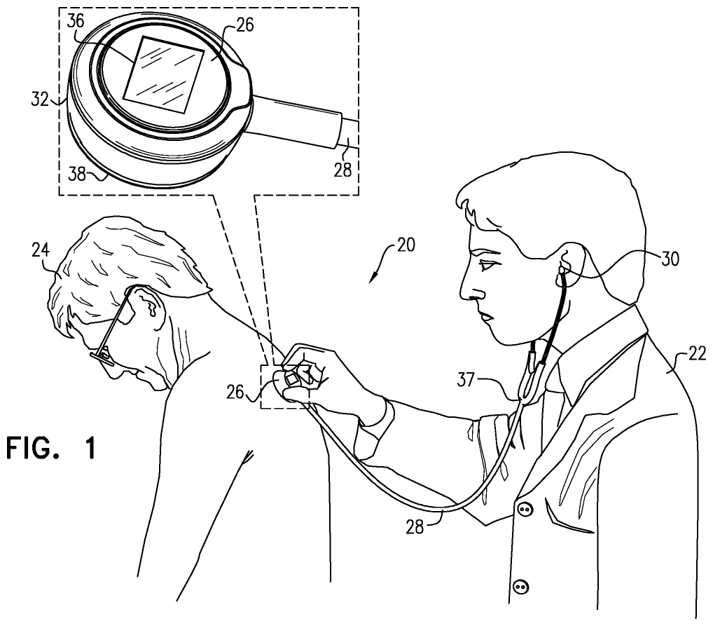

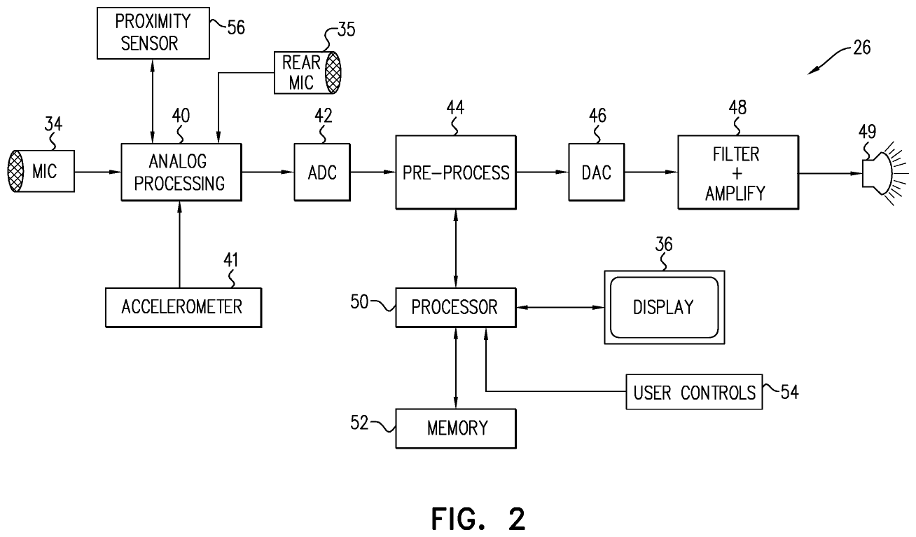

FIG. 1 is a schematic pictorial illustration showing the use of diagnostic apparatus in the form of a digital stethoscope 20, in accordance with an embodiment of the invention. A practitioner 22 brings a head 26 of stethoscope 20 into contact with the body of a patient 24. Head 26 serves as an auscultation pickup unit and contains processing circuitry (shown in FIG. 2) that processes signals acquired by the head in order to generate diagnostic decisions, as described in detail hereinbelow.

Processing circuitry in head 26 may also output an audio signal via a cable 28 extending from head 26 to one or more speakers, which are typically integrated in or acoustically coupled to earphones 30. The two earphones 30 are joined at a spring-loaded junction 37, and thus open apart and fit into the practitioner's ears as would the earphones of a conventional stethoscope. This arrangement is useful in allowing practitioner 22 to listen to the sounds received from the patient's body, as a supplement to the automated diagnostic functions of stethoscope 20 that are described below. Alternatively, these automated diagnostic functions may be carried out entirely without the use of earphones 30.

As shown in the inset, head 26 comprises a case 32, comprising a membrane 38 on its front surface. Case 32 contains an acoustic transducer, such as a suitable microphone (shown in FIG. 2), which senses acoustic waves emitted from the body of patient 24. The microphone may be of any suitable type that is known in the art, for example a piezoelectric sensor or a MEMS (micro-electro-mechanical systems) microphone, which is sensitive not only to audible frequencies, but also to infrasonic frequencies going down to about 5 Hz. Alternatively or additionally, an accelerometer in head 26 (also shown in FIG. 2) can be used to sense low-frequency infrasonic vibrations. On the other side of head 26, a user interface, such as a touch-sensitive display 36, guides practitioner 22 in placing head 26 in contact with appropriate locations on the body of patient 24 for purposes of automated signal acquisition and diagnosis. Display 36 also enables practitioner 22 to control the functions of stethoscope 20 and displays data, such as acoustic signatures of heart and/or breath sounds sensed by microphone 34. Other components and functions of head 26 are described with reference to the figures that follow.

FIG. 2 is a block diagram that schematically illustrate functional elements of stethoscope 20, in accordance with an embodiment of the invention. In the present embodiment, the elements shown in FIG. 2 are assumed to be contained inside head 26, within case 32, with the possible exception of one or more speakers 49 (and possibly user controls 54, as explained below). Alternatively, however, certain processing components may be housed in other parts of stethoscope 20 or in an external processing unit with a suitable communication link (not shown) to head 26. All such alternative embodiments are considered to be within the scope of the present invention.

A microphone 34 is mounted on membrane 38 (FIG. 1) at the front surface of head 26. When the membrane is brought into contact with the patient's body, acoustic waves from the body impinge on the membrane, generating an acoustic signal, which is received by microphone 34. Features of the membrane and microphone are described further in the above-mentioned PCT International Publication WO 2017/141165.

An analog front end 40 performs analog processing functions, including filtering, buffering and amplification of the electrical signals output by microphone 34. Optionally, head 26 also contains a rear microphone 35, which captures background sounds. These background sounds are subtracted from the signals output by microphone 34. The subtraction may be carried out either in analog front end 40 or digitally, for example by adaptive filtering in either the time or frequency domain, following digitization of the signals, as described below.

Head 26 also contains a motion sensor, such as an integrated circuit accelerometer 41, which measures motion of head 26 and low-frequency vibrations of membrane 38. Analog front end 40 processes the signals output by the motion sensor, as well. Accelerometer 41 is typically disposed on the inner side of membrane 38, and may serve at least two functions: both detecting movement of the chest caused by respiration, which causes head 26 to move cyclically at the respiration frequency, and detecting deflections of membrane 38 at vibrational frequencies below the minimum frequency that is detectable by microphone 34. The accelerometer and microphone thus complement one another, in that the accelerometer detects sound at very low frequencies that are not detectable by the microphone, and the microphone detects sound at higher frequencies that are not detectable by the accelerometer.

An analog/digital converter (ADC) 42 digitizes the acoustic and inertial signals, and possibly also other analog inputs. For purposes of audio enhancement and analysis, a digital preprocessing circuit 44 transforms the digitized signals to the frequency domain, for example by computing a short-time Fourier transform (STFT) over successive time windows of the signals. In addition, circuit 44 can perform digital filtering functions, such as noise suppression, and “frequency stretching”: shifting infrasonic frequency components to the audible frequency range, as described in the above-mentioned PCT International Publication WO 2019/048961.

Following these filtering and frequency stretching steps, circuit 44 converts the frequency-domain samples back to the time domain. A digital/analog converter (DAC) 46 converts the stream of processed time-domain samples to analog form. In this manner, practitioner can choose to hear audible versions of the infrasonic frequency components captured by microphone 34 and accelerometer 41, following frequency-stretching to the audible range, in addition to or instead of the audible frequency components themselves that are captured by the microphone. An analog output circuit 48 filters and amplifies the analog signal to generate an electrical audio output to speaker or speakers 49.

A programmable processor 50 receives the stream of samples—in either the time domain or the frequency domain, or both—from digital preprocessing circuit 44. Processor 50 is coupled to a memory 52, which typically comprises non-volatile memory, such as flash memory, containing software or firmware to control the operations of processor 50. In addition, memory 52 typically comprises volatile random-access memory (RAM), which is used by processor 50 to store the digital samples received from circuit 44, as well as to store processing results.

Processor 50 collects the digital signals received by head 26 while the head contacts multiple different locations on the body of patent 24. Processor 50 extracts features of the breath sounds of the patient from the digital signals at each of these locations, and computes a respective local score for each of the locations based on the extracted features. Processor 50 then combines the local scores in order to classify the patient's respiratory condition. Details of this classification process are described below.

In processing the acoustic signals due to the patient's breath sounds, processor 50 may also apply other signals provided by the sensors in head 26. For example, processor may use the motion signal generated by accelerometer 41 in identifying the patient's respiratory cycle and may then apply the identified respiratory cycle in extracting the features of the breath sounds. The respiratory cycle provides timing benchmarks for extracting both time-domain and frequency-domain parameters over multiple respiratory cycle. Using the timing of the respiratory cycle, processor 50 can distinguish between the features of the inspiratory part and expiratory part of the cycle, which can vary among different respiratory conditions.

As another example, processor 50 may identify the patient's heart sounds in the signals provided by microphone 34 and may thus compute the patient's heart rate. Features of the heart rate and/or heart sounds may also be used in classifying the patient's health condition.

In some diagnostic procedures, patient 24 may be asked to vocalize certain sounds while head 26 contacts the patient's body. In this case, microphone 34 captures the sounds that have propagated through the patient's chest, while microphone 35 captures sounds that have propagated through the surrounding air. Processor 50 may compare the digital signals derived from both microphones in order to compute additional features for use in the diagnostic computation.

Processor 50 renders an output to display 36 and/or outputs diagnostic information via a communication link (not shown). In one embodiment, the output indicates whether the patient's respiratory condition is positive, negative, or inconclusive with respect to a particular respiratory illness (such as COVID-19) or possibly two or more illnesses. Additionally or alternatively, the processor may render an acoustic signature to the display, illustrating graphically the spectral features of the patient's respiratory signals. Methods for computation and display of such acoustic signatures are described, for example, in the above-mentioned PCT International Publication WO 2019/048961.

In addition, processor 50 may receive and carry out user instructions, for example in response to finger gestures on the touch screen of display 36. Additionally or alternatively, stethoscope 20 may comprise other user controls 54, such as an on/off switch.

The processing components shown FIG. 2, including analog front end 40, ADC 42, digital preprocessing circuit 44, DAC 46, analog output circuit 48, processor 50 and memory 52, are collectively and individually referred to herein as “processing circuitry.” These components are typically implemented in integrated circuits, as are known in the art, which are mounted together on a printed circuit board within case 32. Alternatively, other implementations of these functional components will be apparent to those skilled in the art after reading the present description and are considered to be within the scope of the present invention. Although FIG. 2 shows a certain arrangement of functional blocks for the sake of conceptual clarity, the functions of at least some of these blocks may be combined into a single integrated circuit chip or, alternatively, split among multiple chips.

Typically (although not necessarily), the functions of stethoscope 20, and specifically of the processing circuitry described above, are powered by a battery (not shown). In order to conserve battery power, it is desirable that at least some of the components of the processing circuitry be powered down automatically when not in use, and then powered up automatically when needed, without requiring practitioner 22 to operate an on/off switch. For this purpose, head 26 comprises a contact sensor, such as a proximity sensor 56, which outputs a proximity signal indicative of contact between the front surface of case 32 and the patient's body. For example, proximity sensor 56 may be an optical sensor, which outputs a signal that is indicative of the proximity of sensor 56 to the patient's skin. Alternatively or additionally, head 26 may comprise other types of contact sensors, such as a strain gauge or other pressure sensor, which measures the pressure of head 26 against the patient's body.

Based on the signal from the contact sensor, processor 50 is able to determine the quality of contact between head 26 and the patient's body. Thus, stethoscope 20 may fully actuate the processing circuitry in head 26 and capture signals only when the contact of quality is sufficient, i.e., with membrane 38 (FIG. 1) firmly contacting the patient's skin. Processor 50 may otherwise power down certain components of the stethoscope when the signal from the contact sensor indicates that the front surface of head 26 is not in contact with the body, and may then power up the components when contact is made.

Additionally or alternatively, based on the quality of the contact between the front surface of head 26 and the body, processor 50 may measure whether practitioner 22 is pressing head 26 against the patient's body with sufficient force, or perhaps too much force, and output an indication of the detected contact quality to practitioner 22. For example, processor 50 may render a graphical and/or alphanumeric output to display 36, indicating that the contact between head 26 and the patient's skin is too weak, or possibly too strong, and may prompt practitioner 22 to modify the contact between head 26 and the patient's body so as to improve the quality of the contact.

In other embodiments, head 26 may comprise other sensors (not shown), which can be used by processor 50 in deriving other physiological parameters for use in assessing the condition of patient 24. These parameters may include, for example, the patient's body temperature, blood oxygen saturation, and/or electrocardiogram.

### Methods for Diagnostic Classification

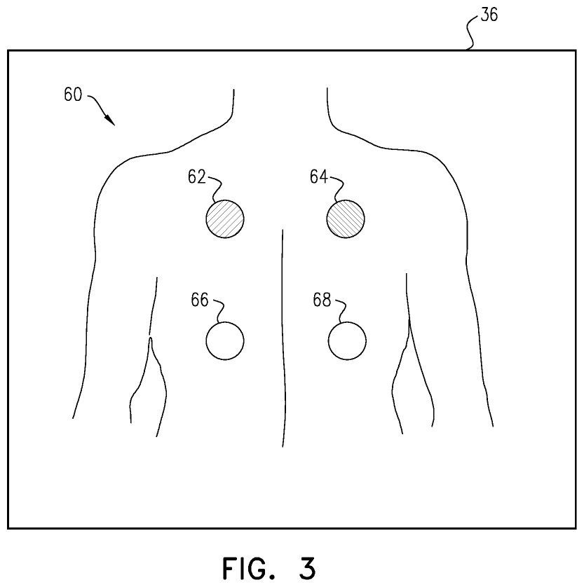

FIG. 3 is a schematic frontal view of display 36 on electronic stethoscope 20 (FIGS. 1 and 2) during a computer-guided diagnostic test, in accordance with an embodiment of the invention. Processor 50 drives the display so as to guide practitioner 22 in placing head 26 in contact with each of a set of locations on the back of patient 24. In this example, the set comprises four locations on the subject's back, at the upper right, lower right, upper left, and lower left. Display 36 presents icons 62, 64, 66, 68 representing the locations and indicating the status of collection of the signals from each of the multiple locations. For example, icon 62 may be presented in one color to indicate that data collection has been completed at the corresponding location, while icon 64 is presented in another color and/or flashes to indicate to practitioner 22 that he should now position head 26 at this next location. Icons 66 and 68 are colored to indicate that their turns will come later in the procedure.

To carry out the test, practitioner 22 places head 26 at a location indicated by one of the icons. Once processor 50 detects that head is in proximity to the body, for example based on the signal from proximity sensor 56, it automatically starts data collection. An animated “processing” icon may be presented on display 36 to indicate that stethoscope 20 is collecting data. The data collection process at each acquisition point typically takes up to 20 seconds.

When data collection from a given location is completed, processor 50 will present a suitable indication on display 36 and will color the corresponding icon (such as icon 62) accordingly. Practitioner 22 then distances head 26 from the patient's back and places it at the next data acquisition location, as indicated on display 26 (for example by icon 64). This procedure is repeated until auscultation data from all four locations have been acquired, and all four icons are colored accordingly on the display.

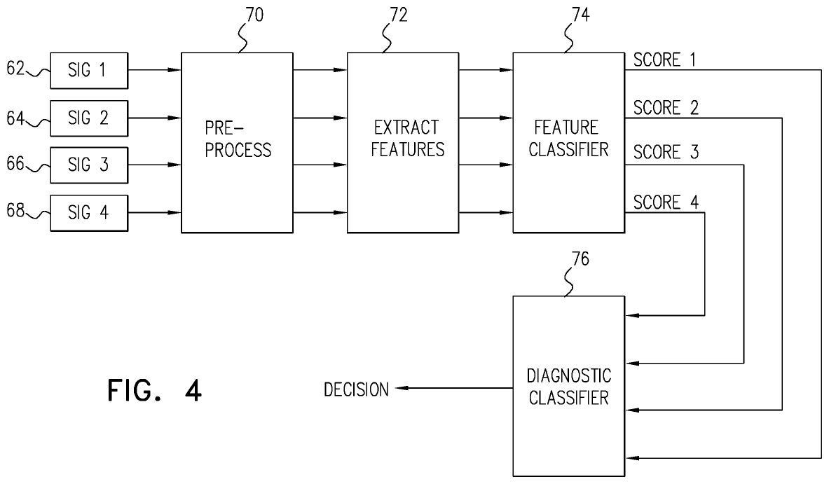

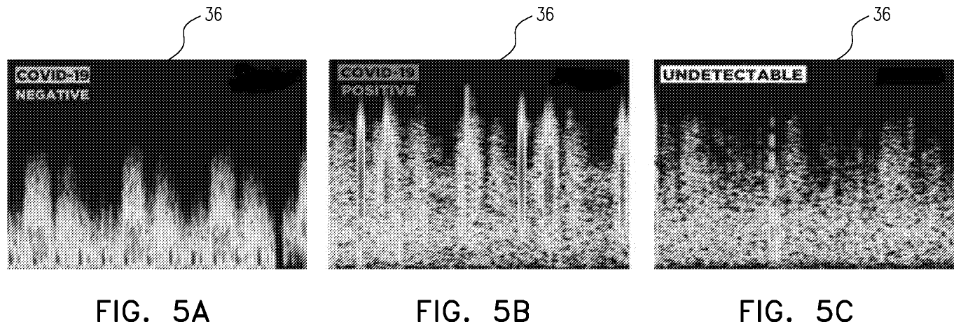

When data acquisition has been completed, processor 50 automatically moves to “analyzing” mode and an appropriate icon is presented on display 36. A method for performing such analysis is described below with reference to FIG. 4. At the conclusion of this analysis, processor 50 presents the results, for example as shown in FIGS. 5A-C.

FIG. 4 is a flow chart that schematically illustrates a method for automated diagnosis of respiratory conditions, in accordance with an embodiment of the invention. In this example, it is assumed that processor 50 is programmed to detect COVID-19, but the principles of the present method may additionally or alternatively be applied, mutatis mutandis, in diagnosing other respiratory ailments. In the present method, processor 50 processes the signals acquired by head 26 to extract features in the time domain, frequency domain, or both, typically including both audible and infrasonic features. These features are then input to a classifier, which is trained to distinguish COVID-19. One specific scheme for feature extraction and classification is described below by way of example. Other choices of features and other types of classifiers will be apparent to those skilled in the art after reading the present description and are considered to be within the scope of the present invention.

The method of FIG. 4 begins with acquiring signals 63, 65, 67 and 69 from four locations on the back of patient 24, such as the locations indicated by icons 62, 64, 66 and 68 in FIG. 3. The signals are indicative of breath sounds and may also include other, complementary physiological signals, as detailed above. The signals are amplified and filtered by analog front end 40 and digital preprocessing circuit 44, in a pre-processing step 70. For purposes of digital processing, ADC 42 samples and digitizes the signals at a high frequency, for example 16,000 Hz. Following digitization, the beginning and end of each signal recording are removed, in order to eliminate noise created by application and removal of head 26 to and from each location on the patient's back. Background noise is removed from the digitized audio signals using the concurrent signals from rear microphone 35. Click noises, which are typically generated by movements of head 26 on the patient's back, are also eliminated. At the conclusion of this preprocessing, the filtered digital signals are down-sampled, for example from 16,000 Hz to 4,000 Hz.

Processor 50 extracts multiple features from each of these down-sampled signals, at a feature extraction step 72. The features calculated for each signal typically relate to both the audible and infrasonic frequency ranges and include both time-domain and frequency-domain parameters. The time-domain parameters include, for example, the average, median, standard deviation, surface under the envelope, entropy, quantile 25%, quantile 75%, and skewness and kurtosis. In the frequency domain, processor 50 calculates several parameters for each of a set of frequency regions, for example seven infrasonic and ten audible frequency regions. The parameters for each frequency region include, for example, the dominant frequency, the magnitude of the dominant frequency, the surface under the curve, and Mel-frequency cepstral coefficients.

Processor 50 inputs the features of each of the four signals into a classifier, at a feature classification step 74. This classifier is implemented in software running on processor 50 and may apply any suitable type of classification algorithm that is known in the art, such as a support-vector machine (SVM) or a convolutional neural network (CNN). At step 74, the classifier computes a respective score for each of the four signals 63, 65, 67, 69, based on the respective features of each signal. These four scores are input to a second classifier running on processor 50, at a diagnostic classification step 76. This classifier outputs a diagnostic decision, indicating whether the patient is positive or negative for COVID-19 (and/or other conditions). When the results of classification step 76 are inconclusive, however, processor 50 may indicate that the patient's condition is undetectable.

Prior to application of the classifiers by stethoscope 20 and implementation by processor 50, the classifiers used at steps 74 and 76 are trained, using features extracted from the breath sounds in a training set acquired from both healthy patients and patients suffering from COVID-19. For example, for each subject included in creating the training set, lung sounds are acquired by stethoscope 26, and a gold-standard chemical COVID-19 test is performed. For COVID-19 positive patients who manifest symptoms, a chest X-ray can be also performed to verify the patients' condition. Following data collection, most of the collected data (for example, 70%) is used in conjunction with the chemical test results to train the classifier model and improve its accuracy. The remainder (30%) of the collected data can be used in testing and verifying the model.

FIGS. 5A, 5B and 5C are schematic frontal views of display 36 on electronic stethoscope 20 showing different results of diagnostic tests carried out using the method of FIG. 4, in accordance with an embodiment of the invention. The display also presents acoustic signatures, which illustrate in graphical form the distribution of spectral energy in the patient's breath sounds. FIG. 5A shows the test output for a patient who was negative for COVID-19, while FIG. 5B shows the test output for a patient who was positive for COVID-19, with characteristic alterations in the acoustic signature. FIG. 5C shows an inconclusive test output, in which the features of the patient's breath sounds were abnormal but did not meet the classification criteria for COVID-19.

It will be appreciated that the embodiments described above are cited by way of example, and that the present invention is not limited to what has been particularly shown and described hereinabove. Rather, the scope of the present invention includes both combinations and subcombinations of the various features described hereinabove, as well as variations and modifications thereof which would occur to persons skilled in the art upon reading the foregoing description and which are not disclosed in the prior art.

1. Medical apparatus, comprising:
an auscultation pickup unit configured to sense acoustic waves emitted from a body of a subject and to output signals in response thereto; and
processing circuitry, which is configured to collect the signals output while the auscultation pickup unit contacts multiple locations on the body of the subject, including a respective signal acquired at each contacted location, to extract from each of the signals features of breath sounds of the subject, to compute multiple local scores including a respective local score for each contacted location based on the features extracted from the respective signal, and to classify a respiratory condition of the subject by combining the multiple local scores.

2. The apparatus according to claim 1, and comprising a user interface, wherein the processing circuitry is configured to drive the user interface so as to guide an operator of the apparatus in placing the auscultation pickup unit in contact with each of the multiple locations.

3. The apparatus according to claim 2, wherein the user interface comprises a display screen, which is configured to display icons representing the multiple locations and indicating a status of collection of the signals from each of the multiple locations.

4. The apparatus according to claim 2, wherein the auscultation pickup unit comprises a contact sensor, which is configured to output a contact signal indicative of contact between the auscultation pickup unit and the body, and wherein the processing circuitry is configured to assess a quality of the contact responsively to the electrical signal and to prompt the operator to modify the contact between the auscultation pickup unit and the body so as to improve the quality of the contact.

5. The apparatus according to claim 2, wherein the multiple locations comprise four locations on a back of the subject, including upper right, lower right, upper left, and lower left locations.

6. The apparatus according to claim 1, wherein the auscultation pickup unit is configured to output the signals in response to both audible and infrasonic acoustic waves emitted from the body.

7. The apparatus according to claim 1, wherein the auscultation pickup unit comprises a motion sensor, which is configured to output a motion signal indicative of movement of the auscultation pickup unit, and wherein the processing circuitry is configured to identify a respiratory cycle of the subject responsively to the motion signal and to apply the identified respiratory cycle in extracting the features.

8. The apparatus according to claim 1, wherein the processing circuitry is configured to identify a heart rate of the subject responsively to the signals and to apply the identified heart rate in extracting the features.

9. The apparatus according to claim 1, wherein the auscultation pickup unit comprises a first acoustic transducer, which is configured to output a first signal in response to the acoustic waves emitted from the body, and a second acoustic transducer, which is configured to output a second signal in response to ambient acoustic waves that are incident on the auscultation pickup unit, and wherein the processing circuitry is configured to extract the features of the breath sounds responsively to a difference between the first and second signals.

10. The apparatus according to claim 9, wherein the processing circuitry is configured to collect the first and second signals while the subject vocalizes one or more predefined sounds, and to apply the collected first and second signals in extracting the features responsively to the vocalized sounds.

11. The apparatus according to claim 1, wherein the extracted features comprise time-domain parameters and frequency-domain parameters of the digital signals.

12. The apparatus according to claim 11, wherein the processing circuitry is configured to compute the frequency-domain parameters for each frequency among a first plurality of audible frequencies and a second plurality of infrasonic frequencies.

13. The apparatus according to claim 1, wherein the processing circuitry is configured to classify the respiratory condition as positive, negative, or inconclusive with respect to a respiratory illness.

14. The apparatus according to claim 13, wherein the processing circuitry is configured to classify the respiratory condition as positive, negative, or inconclusive with respect to COVID-19.

15. A method for medical diagnosis, comprising:
sensing acoustic waves emitted from each of multiple locations on a body of a subject using an auscultation pickup unit, which contact each of the locations, and outputting respective signals in response thereto, including a respective signal acquired at each contacted location;
extracting from the signals features of breath sounds of the subject;
computing multiple local scores including a respective local score for each contacted location based on the features extracted from the respective signal; and
classifying a respiratory condition of the subject by combining the multiple local scores.

16. The method according to claim 15, and comprising guiding an operator, via a user interface of the auscultation pickup unit, in placing the auscultation pickup unit in contact with each of the multiple locations.

17. The method according to claim 16, wherein the user interface comprises a display screen, wherein guiding the operator comprises displaying icons representing the multiple locations on the display screen and indicating a status of collection of the signals from each of the multiple locations.

18. The method according to claim 16, wherein the auscultation pickup unit comprises a contact sensor, which is configured to output a contact signal indicative of contact between the auscultation pickup unit and the body, and wherein the method includes assessing a quality of the contact responsively to the contact signal, and prompting the operator to modify the contact between the auscultation pickup unit and the body so as to improve the quality of the contact.

19. The method according to claim 16, wherein the multiple locations comprise four locations on a back of the subject, including upper right, lower right, upper left, and lower left locations.

20. The method according to claim 15, wherein sensing the acoustic waves comprises outputting the signals from the auscultation pickup unit in response to both audible and infrasonic acoustic waves emitted from the body.

21. The method according to claim 15, wherein the auscultation pickup unit comprises a motion sensor, which is configured to output a motion signal indicative of movement of the auscultation pickup unit, and wherein the method comprises identifying a respiratory cycle of the subject responsively to the motion signal and applying the identified respiratory cycle in extracting the features.

22. The method according to claim 15, and comprising identifying a heart rate of the subject responsively to the signals and applying the identified heart rate in extracting the features.

23. The method according to claim 15, wherein sensing the acoustic waves comprises applying a first acoustic transducer to sense the acoustic waves emitted from the body, and applying a second acoustic transducer to sense ambient acoustic waves that are incident on the auscultation pickup unit, and wherein extracting the features comprises computing the features of the breath sounds responsively to a difference between first and second signals output respectively by the first and second acoustic transducers.

24. The method according to claim 23, wherein sensing the acoustic waves comprises collecting the first and second signals while the subject vocalizes one or more predefined sounds, and applying the collected first and second signals in extracting the features responsively to the vocalized sounds.

25. The method according to claim 15, wherein the extracted features comprise time-domain parameters and frequency-domain parameters of the digital signals.

26. The method according to claim 25, wherein extracting the features comprises computing the frequency-domain parameters for each frequency among a first plurality of audible frequencies and a second plurality of infrasonic frequencies.

27. The method according to claim 15, wherein classifying the respiratory condition comprises identifying the respiratory condition as positive, negative, or inconclusive with respect to a respiratory illness.

28. The method according to claim 27, wherein identifying the respiratory condition comprises classifying the respiratory condition as positive, negative, or inconclusive with respect to COVID-19.
